(19) United States
(12) Patent Application Publication
(10) Pub. No.: US 2017/0157055 A1
(43) Pub. Date: Jun. 8, 2017

(54) PHARMACEUTICAL ABUSE DETERRENT COMPOSITION

(71) Applicants: Jayendrakumar Dasharathlal Patel, Vijapur (IN); Shwetaben Dasharathbhai Patel, Vijapur (IN)

(72) Inventors: Jayendrakumar Dasharathlal Patel, Vijapur (IN); Shwetaben Dasharathbhai Patel, Vijapur (IN)

(73) Assignee: Jayendrakumar Dasharathlal Patel, Vijapur (IN)

(21) Appl. No.: 15/327,224

(22) PCT Filed: Jun. 3, 2015
(86) PCT No.: PCT/IN2015/050041

§ 371 Date: Jan. 18, 2017

(30) Foreign Application Priority Data
Sep. 27, 2014 (IN) 3095/MUM /2014

(51) Int. Cl. A61K 9/28; A61K 9/50

(52) U.S. Cl. CPC A61K 9/28; A61K 9/2806; A61K 9/50

(57) ABSTRACT

An abuse deterrent pharmaceutical composition comprising: (a) an active ingredient; (b) deterrent agent; (c) pH dependent soluble ingredient; and (d) pH modifying agent; wherein deterrent agent is not release in effective amount to produce its intended aversive effect upon correct administration but it release in effective amount to produce its intended aversive effect upon incorrect administration.

### FIELD OF INVENTION

This invention relates to an abuse deterrent pharmaceutical composition, methods of administration, and methods of making the same.

### BACKGROUND OF THE INVENTION

When drugs are used in a manner or amount inconsistent with the medical or social patterns of a culture, it is called drug abuse.

Many pharmaceutical products are sometimes the subject of abuse. There are various routes of administration an abuser may commonly employ to abuse drug formulation. The most common methods include 1) parenteral (e.g. intravenous injection), 2) intranasal (e.g., snorting), and 3) repeated oral ingestion of excessive quantities, for example, of orally administered tablets or capsules. When oral dose of opioid administered as parentally, it is more potent and it produce euphoria. Controlled or immediate release opioid agonist formulations are sometimes crushed or subject to extraction with solvents (e.g., alcohol, water, etc.) by drug abusers to provide the opioid contained therein for immediate release upon oral or parenteral administration.

Because the potential for abuse of prescription drug products has become an important issue for the world, the pharmaceutical industry is striving to develop abuse deterrent formulations in order to reduce the potential for misuse of prescription drugs. Attempts have been made to diminish the abuse potential of orally administered drugs. These attempts generally centered on the inclusion in the oral dosage form of an antagonist, emetic agent or flushing agent which are not orally active but which will substantially produce its therapeutic effect if dosage form is administered after crushing. However, when abuser administer excessive quantity of dosage form, such kind of attempts is not useful because it not release antagonist, emetic agent or flushing agent in therapeutically effective amount.

In particular, patent application no. WO2014085599A1 assign to Accura Pharmaceutical, Inc develop an abuse deterrent pharmaceutical composition comprising: (1) a pharmaceutically active ingredient; (2) an acid soluble ingredient; and (3) a buffering ingredient; wherein the acid soluble ingredient and the buffering ingredient retard release of the pharmaceutically active ingredient when the composition is ingested in excess of an intended dosage.

Patent application no. WO2014145195 A1 assign to Cerovene, Inc develop an oral pharmaceutical composition in unit dosage form comprising: (1) an first portion comprising: an active ingredient and a pH-dependent component, and (2) a second portion comprising a pH-raising agent.

To the best of our knowledge, there is still a need to develop a pharmaceutical abuse deterrent dosage form that significantly reduces the potential for improper administration of drug or drugs which has high potential for abuse.

### OBJECTS OF THE INVENTION

It is an object of present invention to provide an abuse deterrent pharmaceutical composition that decreases improper administration of drug or drugs contained therein.

It is an object of present invention to provide an abuse deterrent pharmaceutical composition having a pH dependant solubility feature and pH modifying feature to release deterrent agent such that, deterrent agent not produces any intended aversive effect in human subject when dosage form administered correctly but it may produce intended aversive effect in human subject when dosage form administered incorrectly.

It is an object of present invention to provide an abuse deterrent pharmaceutical composition which is therapeutically effective if administered correctly.

It is an object of present invention to provide an abuse deterrent pharmaceutical composition that may not produces any intended aversive effect or may produce mild aversive effect in physically dependent human subject if administered correctly.

It is an object of present invention to provide an abuse deterrent pharmaceutical composition that may produce intended aversive effect without substantial delay or after certain period of time in physically dependent human subject if administered incorrectly.

It is an object of present of invention to provide an abuse deterrent pharmaceutical composition that is suitable to treat acute or chronic physical condition in human subjects while reducing its misuse by any route of administration.

It is an object of present invention to provide an abuse deterrent pharmaceutical composition that may be adapted for oral administration 1-8 times a day, such as 1-6 times daily, including 1-4 times, 1-3 times, 1-2 times or 1 times daily.

It is an object of present invention to provide a method of manufacturing for an abuse deterrent pharmaceutical composition which has less abuse potential

### SUMMARY OF INVENTION

In preferred embodiment, an abuse deterrent pharmaceutical composition comprising:
a. an active ingredient;
b. deterrent agent;
c. pH dependent soluble ingredient; and
d. pH modifying agent;
wherein deterrent agent is not release in effective amount to produce its intended aversive effect upon correct administration to an individual but it release in effective amount to produce its intended aversive effect upon incorrect administration to an individual.

In some embodiment, deterrent agent contained within matrix of pH dependent soluble ingredient or coated by pH dependent soluble ingredient.

In present invention, an abuse deterrent pharmaceutical composition may be formulated having a pH dependant solubility feature and pH modifying feature to release deterrent agent such that, under normal dosing conditions, when a prescribed dose is ingested, amount of pH modifying agent added in prescribed dose is not in sufficient amount to modify pH of gastrointestinal fluid at desire level to release deterrent agent in effective amount in gastrointestinal fluid but when ingested in excess of prescribed or therapeutic dose (i.e., two or three or more times), amount of pH modifying agent of multiple dose units is in sufficient amount to modify pH of gastrointestinal fluid at desire level to release deterrent agent in gastrointestinal fluid in effective amount to produce its intended aversive effect.

Dosage form prepared according to invention when undergo crushing, matrix or coating of pH dependent soluble ingredient surrounding deterrent agent is break and release the deterrent agent or when undergo extraction with solvent, such as for example alcohol, matrix or coating of pH dependent soluble ingredient surrounding deterrent agent is dissolve in solvent and release the deterrent agent. Therefore, not only drug extracts or dissolved in solvent but deterrent agent is also extract or dissolved in solvent, which produce resistance against drug abuse by any individual.

Thus, deterrent agent may release upon ingestion of excessive dose or upon physical alteration or during extraction with solvent. Hence, an abuse deterrent pharmaceutical composition prepared according to invention is suitable to reduce abuse of drug by any route, for example, by oral, by nasal or by parenteral.

### DETAILED DESCRIPTION OF INVENTION

The term “abuse” is intended to denote the use of a drug or drugs in order to induce euphoria, i.e. the use is not intended to cure a disease or alleviate disease symptoms, but rather for obtaining intoxication.

An abuse deterrent pharmaceutical composition means composition that discourages the intention of incorrect administration of drug or drugs contained therein for obtaining intoxication.

The term “incorrect administration” is used herein to refer the administration of drug or drugs at dose higher than therapeutically effective dose or prescribed dose or intended dose or administration of drug by any route after physical alteration of dosage form or administration of drug after extraction from dosage form Administration of dose higher than therapeutically effective dose or prescribed dose or intended dose means administration of dosage units in multiple numbers at a time or within certain time period, e.g., administration of 2 times or 3 times or more times dose than usually prescribe dose or intended dose.

According to an invention, an abuse deterrent pharmaceutical composition releases the drug or drugs in immediate release form or in controlled release form or in combination of both. The term “controlled release” is used in present invention to refer “delayed release”, “extended release”, “prolong release”, “long-acting”, “modified release”, “slow release”, “sustained release”, “pulsatile release”, “time release” and the like, all of which are understood to refer to a release which is later or slower than “immediate release” or “release at any rate after certain period of time.”

According to an embodiment, an abuse deterrent pharmaceutical composition of present invention comprises a unit dose or therapeutic effective dose of drug or drugs in combination with deterrent agent. The term “deterrent agent” is used herein to refer agent or ingredient that discourages the intention of incorrect administration of dosage form by producing aversive effect. Aversive effect means effect produced by deterrent agent that discourages the intention of incorrect administration of dosage form for obtaining intoxication. Aversive effect produced by deterrent agent include decrease or block the action of drug substance or produce feeling of dislike, feeling of uncomfortable or any unpleasant effect.

In preferred embodiments, an abuse deterrent pharmaceutical composition of the present invention is designed that produce its intended therapeutic effect in an individual upon correct administration. Under correct administration, the inventive formulations may not release deterrent agent in effective amount to produce intended aversive effect. But, in the case of intentional incorrect administration of dosage form, such as where an individual would consume excess dosage units of an abused drug or administration of dosage form after physical alteration or administration of drug after extraction from dosage form to experience a euphoric effect, a deterrent agent would be release in effective amount and produce its intended aversive effect in an individual. In this way, the inventive formulation may work as a deterrent from abusing the drug substance for the purpose of achieving the euphoric effect. Yet the patient who uses the inventive formulation as directed will receive the desired therapeutic treatment without any intended aversive effect or with mild aversive effect.

In present invention, “not release in effective amount” means no release or release in an amount that not produces any intended effect or produce mild effect. In present invention, “deterrent agent not release in effective amount” means no release of deterrent agent or release of deterrent agent in an amount that not produces any intended aversive effect or produces mild aversive effect. In present invention, “deterrent agent release in effective amount” means release of deterrent agent in an amount that produces intended aversive effect.

In preferred embodiment, an abuse deterrent pharmaceutical composition comprising:
a. an active ingredient;
b. deterrent agent;
c. pH dependent soluble ingredient; and
d. pH modifying agent;
wherein deterrent agent is not release in effective amount to produce its intended aversive effect upon correct administration to an individual but it release in effective amount to produce its intended aversive effect upon incorrect administration to an individual.

In preferred embodiment, an abuse deterrent pharmaceutical composition can be in unit dosage form. The term “unit dosage form” refers to dosage form comprising therapeutic effective dose of drug for administration to a subject. Examples of unit dosage forms include but are not limited to tablets, capsules, suppositories, multiparticulates packed in unit dose container or compressed to form tablet. Multiparticulates form such as microtablets or minitablets, micropellets, granules, spheres, spheroids, beads, pellets, particles, microparticles, nanoparticles and like.

In preferred embodiment, deterrent agent contained within matrix of pH dependent soluble ingredient or coated by pH dependent soluble ingredient. In preferred embodiment, pH modifying agents are acidifying agent or basifying agent. In some embodiment, pH modifying agent may also contained within matrix of pH dependent soluble ingredient or coated by pH dependent soluble ingredient.

In some embodiment, an abuse deterrent pharmaceutical composition may be formulated having a pH dependant solubility feature and pH modifying feature to release deterrent agent such that, deterrent agent may not produces any intended aversive effect in human subject when dosage form administer correctly but it may produce intended aversive effect in human subject when dosage form administer incorrectly. In some embodiment, release of a deterrent agent from dosage form is governed by pH dependent soluble agent and/or pH modifying agent. Under normal dosing conditions, when a prescribed dose is ingested, amount of pH modifying agent added in prescribed dose is not in sufficient amount to modify pH of gastrointestinal fluid at desire level to release deterrent agent in effective amount in gastrointestinal fluid but when ingested in excess of prescribed or therapeutic dose (i.e., two or three or more times), amount of pH modifying agent of multiple dose units are in sufficient amount to modify pH of gastrointestinal fluid at desire level to release deterrent agent in gastrointestinal fluid in effective amount to produce its intended aversive effect. Thus, an abuse deterrent pharmaceutical composition may deter an abuse of drug or drugs when administer in excessive dose units.

In some embodiment, when dosage form undergo crushing, matrix or coating of pH dependent soluble ingredient surrounding deterrent agent is break and release the deterrent agent or dosage form when undergo extraction with solvent, such as for example alcohol, coating of pH dependent soluble ingredient surrounding deterrent agent is dissolve in solvent and release the deterrent agent. Therefore, not only drug extracts or dissolved in solvent but deterrent agent is also extract or dissolved in solvent, which produce resistance against drug abuse by any individual. Thus, an abuse deterrent pharmaceutical composition may deter an abuse of drug or drugs from any route after physical alteration of dosage form or extraction of drug from dosage form.

Hence, an abuse deterrent pharmaceutical composition according to invention is suitable to deter an abuse of drug from any route, for example, by oral, by nasal or by parenteral.

In preferred embodiment, an abuse deterrent pharmaceutical composition comprising:
a. an active ingredient;
b. deterrent agent;
c. pH dependent soluble ingredient; and
d. pH modifying agent;
   wherein pH dependent soluble ingredient and pH modifying agent allow to release deterrent agent in effective amount to produce its intended aversive effect when dosage form is ingested in excess of an intended dosage.

In preferred embodiment, pH modifying agents are acidifying agent or basifying agent or both. In some embodiment, pH modifying agent may also contained within matrix of pH dependent soluble ingredient or coated by pH dependent soluble ingredient.

In present invention, an abuse deterrent pharmaceutical composition may be formulated having a pH dependant solubility feature and pH modifying feature to release deterrent agent such that, under normal dosing conditions, when a prescribed dose is ingested, amount of pH modifying agent added in prescribed dose is not in sufficient amount to modify pH of gastrointestinal fluid at desire level to release deterrent agent in effective amount in gastrointestinal fluid but when ingested in excess of prescribed or therapeutic dose (i.e., two or three or more times), amount of pH modifying agent of multiple dose units is in sufficient amount to modify pH of gastrointestinal fluid at desire level to release deterrent agent in gastrointestinal fluid in effective amount to produce its intended aversive effect. Thus, an abuse deterrent pharmaceutical composition of the present invention may be designed with one or more components to control release of deterrent agent from dosage form when ingested in excessive amount.

In some embodiments, an abuse deterrent pharmaceutical composition may be designed with a pH modifying feature and a pH dependent solubility feature. A pH dependent solubility feature may impact release of deterrent agent depending on the pH of the gastrointestinal environment. A pH dependent solubility feature may be provided by inclusion of one or more pH dependent soluble ingredients in the pharmaceutical composition. A pH modifying feature support pH dependent solubility feature by modifying the pH of the gastrointestinal environment based on whether the pharmaceutical composition is taken at an appropriate dosage amount or in excess. Thus, pH modifying feature may also impact release of deterrent agent from dosage form. A pH modifying feature may be provided by inclusion of one or more acidifying and/or basifying ingredient in the pharmaceutical composition.

In one embodiment, an abuse deterrent pharmaceutical composition in unit dosage form comprising:
a. a drug or drugs;
b. at least one deterrent agent contained within matrix of pH dependent soluble ingredient (A) or coated by pH dependent soluble ingredient (A), particularly ingredient which is soluble at pH of stomach fluid to retard the release of deterrent agent in intestinal fluid;
c. pH modifying basic agent; and
d. coat the step (b) deterrent agent with pH dependent soluble ingredient (B) which is soluble at pH of intestinal fluid to retard release of deterrent agent in stomach fluid;
   wherein pH dependent soluble ingredient and pH modifying agent allow to release deterrent agent in effective amount to produce its intended aversive effect when dosage form is ingested in excess of an intended dosage.

In another embodiment, an abuse deterrent pharmaceutical composition in unit dosage form comprising:
a. a drug or drugs;
b. at least one deterrent agent contained within matrix of pH dependent soluble ingredient (A) or coated by pH dependent soluble ingredient (A), particularly ingredient which is soluble at pH of stomach fluid to retard the release of deterrent agent in intestinal fluid;
c. pH modifying basic agent; and
d. coat the step (b) deterrent agent with pH dependent soluble ingredient (B) which is soluble at pH of intestinal fluid to retard release of deterrent agent in stomach fluid;
   wherein said dosage form when ingested as per intended dosage, pH modifying agents are not in sufficient amount in intended dosage unit(s) to modify gastrointestinal fluid at desire level to release deterrent agent in effective amount in gastrointestinal fluid but when dosage form is ingested in excess of intended dosage unit(s), pH modifying basic agent of multiple dosage units increase the pH of stomach fluid to pH of intestinal fluid (desire pH level) which solubilize pH dependent soluble ingredient (B) of step (d) and furthermore continuous secretion of gastric juice inside the stomach gradually decrease pH of stomach fluid below pH 5.5 (desire pH level) and hence pH dependent soluble ingredients (A) surrounding deterrent agent is dissolve which allow release of deterrent agent in effective amount in gastrointestinal fluid to produce its intended aversive effect.

In one embodiment, an abuse deterrent pharmaceutical composition in unit dosage form comprising:
a. a drug or drugs;
b. at least one deterrent agent contained within matrix of pH dependent soluble ingredient (A) or coated by pH dependent soluble ingredient (A), particularly ingredient which is soluble at pH of stomach fluid to retard the release of deterrent agent in intestinal fluid;
c. pH modifying acidic agent; and
d. coat the step (b) deterrent agent and step (c) pH modifying acidic agent with pH dependent soluble ingredient (B) which is soluble at pH of intestinal fluid to retard release of deterrent agent and pH modifying acidic agent in stomach;
   wherein pH dependent soluble ingredient and pH modifying agent release deterrent agent in effective amount to produce its intended aversive effect when dosage form is ingested in excess of an intended dosage.

In another embodiment, an abuse deterrent pharmaceutical composition in unit dosage form comprising:
a. a drug or drugs;
b. at least one deterrent agent contained within matrix of pH dependent soluble ingredient (A) or coated by pH dependent soluble ingredient (A), particularly ingredient which is soluble at pH of stomach fluid to retard the release of deterrent agent in intestinal fluid;
c. pH modifying acidic agent; and
d. coat the step (b) deterrent agent and step (c) pH modifying acidic agent with pH dependent soluble ingredient (B) which is soluble at pH of intestinal fluid to retard release of deterrent agent and pH modifying acidic agent in stomach;
   wherein said dosage form when ingested as per intended dosage, pH dependent soluble ingredient (B) is dissolve in intestinal fluid and release the step (c) pH modifying acidic agent and step (b) deterrent agent in intestinal fluid, however pH modifying acidic agent is not in sufficient amount in intended dosage unit(s) to decrease pH of intestinal fluid to pH of stomach fluid to solubilize ingredients (A) surrounding deterrent agent but when dosage form is ingested in excess of intended dosage unit(s), pH modifying acidic agent of multiple dosage units decreases pH of intestinal fluid to pH of stomach fluid (desire level) and hence pH dependent soluble ingredients (A) surrounding deterrent agent is dissolve which allow the release of deterrent agent in intestinal fluid in effective amount to produce its intended aversive effect.

In one embodiment, an abuse deterrent pharmaceutical composition in unit dosage form comprising:
a. a drug or drugs
b. at least one deterrent agent contained within matrix of pH dependent soluble ingredient (A) or coated by pH dependent soluble ingredient (A), particularly ingredient which is soluble at pH of stomach fluid to retard the release of deterrent agent in intestinal fluid;
c. pH modifying acidic agent;
d. pH modifying basic agent; and
e. coat the step (b) deterrent agent and step (c) pH modifying acidic agent with pH dependent soluble ingredient (B) which is soluble at pH of intestinal fluid to retard release of deterrent agent and pH modifying acidic agent in stomach fluid;
   wherein pH dependent soluble ingredient and pH modifying agent allow to release deterrent agent in effective amount to produce its intended aversive effect when dosage form is ingested in excess of an intended dosage.

In one embodiment, an abuse deterrent pharmaceutical composition in unit dosage form comprising:
a. a drug or drugs
b. at least one deterrent agent contained within matrix of pH dependent soluble ingredient (A) or coated by pH dependent soluble ingredient (A), particularly ingredient which is soluble at pH of stomach fluid to retard the release of deterrent agent in intestinal fluid;
c. pH modifying acidic agent;
d. pH modifying basic agent; and
e. coat the step (b) deterrent agent and step (c) pH modifying acidic agent with pH dependent soluble ingredient (B) which is soluble at pH of intestinal fluid to retard release of deterrent agent and pH modifying acidic agent in stomach fluid;
   wherein said dosage form when ingested as per intended dosage, pH modifying agents are not in sufficient amount in intended dosage unit(s) to modify gastrointestinal fluid at desire level to release deterrent agent in effective amount in gastrointestinal fluid but when dosage form is ingested in excess of intended dosage unit(s), pH modifying basic agent of multiple dosage units increase the pH of stomach fluid to pH of intestinal fluid and hence pH dependent soluble ingredient (B) surrounding step (b) and step (c) ingredients are dissolve in stomach fluid resulting into release of pH modifying acidic agent of multiple dosage units which decrease pH of stomach fluid below 5.5 and hence pH dependent soluble ingredient (A) surrounding deterrent agent is dissolve and deterrent agent is release in gastrointestinal fluid in effective amount to produce its intended aversive effect.

As a result, an abuse deterrent pharmaceutical composition may be formulated having a pH dependant solubility feature and pH modifying feature such that, under normal dosing conditions, when a prescribed dose is ingested, amount of pH modifying agent of prescribed dose unit(s) is not in sufficient amount to modify pH of gastrointestinal fluid at desire level to release deterrent agent in effective amount to produce its intended aversive effect, but when dose is ingested in excess of prescribed dose or intended dosage, amount of pH modifying agent of multiple dose units is in sufficient amount to modify pH of gastrointestinal fluid at desire level to release deterrent agent in effective amount to produce its intended aversive effect. In other word, the combination of pH dependant solubility feature and pH modifying feature may contribute to the abuse deterrent aspect of the pharmaceutical composition.

### Components of an Abuse Deterrent Pharmaceutical Composition:

### Active Substance—

In preferred embodiment, an abuse deterrent pharmaceutical composition comprising drug or drugs. The terms “drug”, “active substance”, “active ingredient”, “active agent”, “drug substance”, “pharmacologically active agent” and “physiologically active agent” or “drug substance in physiologically active form” are used interchangeably herein to refer to a chemical compound that induces a desired pharmacological or physiological effect or biological activity. The terms also encompass pharmaceutically acceptable derivatives of those active agents specifically mentioned herein, including, but not limited to, salts, solvates, polymorphs, hydrates, complexes with one or more molecules, prodrug, active metabolites, analogs, and the like. When the terms “drug”, “active agent”, “pharmacologically active agent” and “physiologically active agent” or “drug substance in physiologically active form” are used, or when a particular drug, for example oxycodone, is identified, it is to be understood as including the active agent as well as pharmaceutically acceptable salts, solvates, polymorphs, hydrates, complexes with one or more molecules, prodrugs, active metabolites, homologue and analogs.

In some embodiment, the drug substance is added in dosage form in powder form, in amorphous form, in crystalline form, in micronized form, in complexes with one or more molecules, in combination of two or more active substance or any combination of thereof

In some embodiments, the active ingredient include, but are not limited to analgesics, anti-inflammatory agents, anti-helminthics, anti-arrhythmic agents, anti-asthma agents, anti-bacterial agents, anti-viral agents, anti-coagulants, anti-dementia agents, anti-depressants, anti-diabetics, anti-epileptics, anti-fungal agents, anti-gout agents, anti-hypertensive agents, anti-malarials, anti-migraine agents, anti-muscarinic agents, anti-neoplastic agents, immunosuppressants, antiprotozoal agents, anti-thyroid agents, anti-tussives, anxiolytics, sedatives, hypnotics, neuroleptics, neuroprotective agents, β-blockers, cardie inotropic agents, cell adhesion inhibitors, corticosteroids, cytokine receptor activity modulators, diuretics, anti-Parkinson's agents, gastro-intestinal agents, histamine H-receptor antagonists, keratolyses, lipid regulating agents, muscle relaxants, nitrates and other anti-anginal agents, non-steroid anti-asthma agents, nutritional agents, opioid analgesics, sex hormones, stimulants and anti-erectile dysfunction agents; and salts, esters, and mixtures thereof.

In preferred embodiments, an active drug substance is associated with abuse syndromes and the active drug substance may, thus for example, be selected from opioids, opiates, CNS depressants or sedative, anxiolytics, narcotic, tranquilizers, barbiturates, hormones, CNS stimulants, cannabinoids, nicotine-like compounds, glutamate antagonists, N-methyl-D-aspartate (NMDA) antagonists or drugs that can cause psychological and/or physical dependence. Drugs that are preferred include those classified as Schedule I, II, III, IV and V drugs based upon the substance's medicinal value, harmfulness, and potential for abuse or addiction under the control substance act.

An abuse deterrent pharmaceutical composition according to the invention is very particularly suitable for preventing abuse of active drug substances includes, but not limited to, are:

1-(1-Phenylcyclohexyl)pyrrolidine, 1-(2-Phenylethyl)-4-phenyl-4-acetoxypiperidine, 1-[1-(2-Thienyl)-cyclohexyl]piperidine, 1-[1-(2-Thienyl)cyclohexyl]pyrrolidine, 1-Methyl-4-phenyl-4-propionoxy-piperidine, 1-Phenylcyclohexylamine, 1-Piperidinocyclohexanecarbonitrile, 2,5-Dimethoxy-4-ethylamphetamine, 2,5-Dimethoxyamphetamine, 2C-B-(4-bromo-2,5-dimethoxypenethylamine), 2C-D (2,5 -dimethoxy-4-methylphenethylamine), 2C-I (4-iodo-2,5-dimethoxy-phenethylamine), 2C-T-2 (2,5-dimethoxy-4-ethylthiophenethylamine), 2C-T-4 (2,5-dimethoxy-4-isopropyl thiophenethylamine), 2C-T-7 (2,5-dimethoxy-4-(n)-propylthiopenethylamine), 3,4-Methylene-dioxymethamphetamine, 3,4,5 -Trimethoxyamphetamine, 3,4-Methylenedioxyamphetamine, 3,4-Methylenedioxy-N-ethylamphetamine, 3-Methylfentanyl, 3-Methylthiofentanyl, 4-Bromo-2,5-dimethoxyamphetamine, 4-Bromo-2,5-dimethoxyphenethylamine, 4-Methoxyamphetamine, 4-Methyl-2,5-dimethoxyamphetamine, 4-Methylaminorex (cis isomer), 5 -MeO-DMT (5 -Methoxy-N,N-diisopropyltryptamine), 5-MeO-DMT (5-Methoxy-N,N-dimethyltryptamine), 5-Methoxy-3,4-methylenedioxyamphetamine, Acetorphin, Acetorphine, Acetyl-alpha-methylfentanyl, Acetyl-alpha-methylfentanyl, Acetyldihydrocodeine, Acetylmethadol, Acetylmethadol, aniloridine, Alfentanil, Allobarbital, Allylprodin, Allylprodine, Alphacetylmethadol except levo-alphacetylmethadol, Alpha-ethyltryptamine, Alphameprodine, Alphamethadol, Alphamethadol, Alpha-Methylfentanyl, Alpha-Methylthiofentanyl, Alphaprodine, Alprazolam, Amfepramon, Amfetaminil, Amineptin, Aminorex, Amobarbital, amfepramone, Amphetamine, amphetaminil, Amylnitrit (all isomers of the amyl group), Anabolic steroids, Anileridine, apocodeine, Aprobarbital, Barbital, Barbituric acid derivative, BDB (3,4-methylenedioxyphenyl)-2-butanamine), Benzethidin, Benzethidine, Benzoylecgonine, Benzphetamine, Benzphetamine, Benzylmethylketon, Benzylmorphine, Betacetylmethadol, Beta-Hydroxy-3-methylfentanyl, Beta-Hydroxyfentanyl, Betameprodine, Betameprodine, Betamethadol, Betaprodine, Bezitramide, Boldenone, Brolamfetamin, Bromazepam, Brotizolam, Bufotenine, Buprenorphine, Butabarbital, Butalbital, Butobarbital, Butorphanol, BZP (A 2)(1-benzylpiperazin), Camazepam, Cannabis, Carfentanil, carfentanyl, Catha edulis, Cathine, Cathinone, Chloral betaine, Chloral hydrate, Chlordiazepoxide, Chlorhexadol, Chlorotestosterone (same as clostebol), Chlorphentermine, Clobazam, Clonazepam, Clonitazene, Clorazepate, Clortermine, Clostebol, Clotiazepam, Cloxazolam, Coca Leaves, Cocaine, Codeine, Codeine & isoquinoline alkaloid, Codeine methylbromide, Codeine-N-oxide, Codoxim, cyclorphan, Cyclobarbital (Hexemal NFN), cyclazocine, Cyprenorphine, Dehydrochlormethyltestosterone, Delorazepam, Desomorphine, Dexamfetamine, dexamphetamine, dexmethylphenidate, Dexfenfluramine, Dextromoramide, dextromethorphan, Dextropropoxyphene, dezocine, Diacetylmorphine, diamorphone, Diampromide, diapromide, Diazepam, Dichloralphenazone, Diethylpropion, Diethylthiambutene, Diethyltryptamine, Difenoxin, Dihydrocodeine, Dihydroetorphine, Dihydromorphine, Dihydrotestosterone, dimephetanol, Dimenoxadol, Dimepheptanol, Dimethylthiambutene, Dimethyltryptamine, Dioxaphetyl butyrate, Diphenoxylate, Dipipanone, Diprenorphine, Dronabinol, Drostanolone, Drotebanol, Ecgonine, eptazocine, Estazolam, Ethchlorvynol, Ethinamate, ethoheptazine, Ethyl loflazepate, Ethylestrenol, Ethylmethylthiambutene, Ethylmorphine, Eticyclidin, Etilamfetamine, Etonitazene, Etorphine, Etoxeridine, Etryptamine, Fencamfamin, Fenethylline, Fenetylline, Fenfluramine, Fenproporex, Fentanyl, Fludiazepam, Flunitrazepam, Fluoxymesterone, Flurazepam, Formebolone, Fungi and Spores of the species PsilocypeSemilanceata, Furethidine, Gammahydroxybutanic acid, Glutethimide, Halazepam, Hallucinogens, Haloxazolam, Heroine, Hydrocodone, Hydrocodone & isoquinoline alkaloid, Hydromorphinol, Hydromorphone, Hydroxypethidine, Ibogaine, Isobutylnitrit, Isomethadone, Ketamine, Ketazolam, Ketobemidone, lefetamine, Levamfetamine, levallorphan, Levo-alphacetylmethadol, Levo-methamphetamine, Levomethorphan, Levomoramide, Levophenacylmorphan, Levorphanol, lisdexamphetamine, lofentanil, loperamide, Loprazolam, Lorazepam, Lormetazepam, Lysergic acid, Lysergic acid amide, Lysergic acid diethylamide, Marijuana, Mazindol, MBDN(N-methyl-1-(3,4-methylenedioxyphenyl)-2-butanamine), mCPP (1-(3-chlorphenyl)piperazine), Mebutamate, Mecloqualone, Medazepam, Mefenorex, MeOPP (1-(4-methoxyphenyl)piperazine), Meperidine intermediate, Meprobamate, meptazinol, Mescaline, Mesocarb, Mesterolone, Metamfetamine, Metazocine, Methadone, Methadone intermediate, Methamphetamine, Methandienone, Methandranone, Methandriol, Methandrostenolone, Methaqualone, Methcathinone, Methenolone, Methohexital, Methyldesorphine, Methyldihydromorphine, Methylphenidate, Methylphenobarbital (mephobarbital), Methyltestosterone, Methyprylone, Metopone, Mibolerone, Midazolam, Modafinil, Moramide-intermediate, Morpheridine, Morphine, morphine 3-glucuronide, morphine 6-glucuronide, Morphine methylbromide, Morphine methylsulfonate, Morphine-N-oxide, Myrophine, N,N-Dimethylamphetamine, Nabilone, nalbuphine, Nalorphine, Nandrolone, narccine, N-Ethyl-1-phenylcyclohexylamine, N-Ethyl-3-piperidyl benzilate, N-Ethylamphetamine, N-Hydroxy-3,4-methylenedioxyamphetamine, Nicocodeine, Nicocodine, Nicodicodine, Nicomorphine, Nimetazepam, Nitrazepam, N-Methyl-3-piperidyl benzilate, Noracymethadol, Norcodeine, Nordiazepam, Norethandrolone, Norlevorphanol, Normethadone, Normorphine, Norpipanone, ohmefentanyl, Opium, Oxandrolone, Oxazepam, Oxazolam, oxycodeine, Oxycodone, Oxymesterone, Oxymetholone, Oxymorphone, papavereturn, Para-Fluorofentanyl, Parahexyl, Paraldehyde, pethidine,Pemoline, Pentazocine, Pentobarbital, Petrichloral, Peyote, Phenadoxone, Phenampromide, Phenazocine, Phencyclidine, Phendimetrazine, Phenmetrazine, Phenobarbital, Phenomorphan, Phenoperidine, Phentermine, Phenylacetone, Pholcodine, Piminodine, Pinazepam, Pipradrole, Piritramide, PMMA (paramethyxymethyl amphetamine), Prazepam, prodine, Proheptazine, promedol, Properidine, Propiram, propheptazine, propoxyphene, Psilocybine, Psilocyn, Pyrovalerone, Quazepam, Racemethorphane, Racemoramide, Racemorphane, Remifentanil, Salvia divinorum, Salvinorin A, Secobarbital, Secobarbital, Sibutramine, SPA, Stanolone, Stanozolol, Sufentanil, Sulfondiethylmethane, Sulfonethylmethane, Sulfonmethane, Talbutal, Tapentadol, Temazepam, Tenamfetamin, Testolactone, Testosterone, Tetrahydrocannabinols, Tetrazepam, TFMPP (1-(3-triflourmethylphenyl)piperazine), Thebacon, Thebaine, Thiamylal, Thiofentanyl, Thiopental, Tiletamine, Tramadol, Zolazepam, Zolazepam, Tilidine, Trenbolone, Triazolam, Trimeperidine, Vinbarbital, Zaleplon, Zipeprol, Zolpidem, Zopiclon.

In one embodiment of present invention, the drug substance may also include new chemical entity for which the amount of information is limited. In such cases, the dosage form regimen needs to evaluate based on preclinical and clinical trials.

The above mentioned active drug substances may also be in the form of pharmaceutically acceptable salts, uncharged or charged molecules, molecular complexes, solvates or anhydrates thereof, and, if relevant, isomers, enantiomers, racemic mixtures, and mixture thereof.

In some embodiments, a dosage form contains an appropriate amount of drug to provide a therapeutic effect.

### Deterrent Agent—

In preferred embodiment, an abuse deterrent pharmaceutical composition comprises at least one deterrent agent.

The term “deterrent agent” is used herein to refer agent or ingredient that discourages the intention of incorrect administration of dosage form by producing aversive effect. Aversive effect means effect produced by deterrent agent that discourages incorrect administration of dosage form for obtaining intoxication. Aversive effect produced by deterrent agent include decrease or block the action of drug substance or produce feeling of dislike, feeling of uncomfortable or any unpleasant effect.

In one embodiment, deterrent agent is present in therapeutically effective amount in single dose unit. In another embodiment, deterrent agent is not present in therapeutically effective amount in single dose unit.

In some embodiment, deterrent agent may not produce any intended aversive effect or produce mildly aversive effect in human subject when dosage form administered correctly but it may produce intended aversive effect in human subject when dosage form administered incorrectly.

According to invention, deterrent agent suitable according to present invention include, but not to be limited, antagonist agent, emetic agent, flushing agent, irritating agent, burning agent and like. However, deterrent agent as describe here are for information, which not limit the scope of invention, i.e. agent other than described here may also be used as deterrent agent, for instance, agent which act according to definition of deterrent agent as described above.

In some embodiment, deterrent agent like antagonist agent, emetic agent or flushing agent are added in physiologically active form or in pharmacologically active form and in pharmaceutically acceptable form in dosage form including, but not limited to, free base, free acid, salts, solvates, hydrates, complexes with one or more molecules, prodrugs, active metabolites, and analogs. Free base or free acid forms of deterrent agent have pH dependent solubility in aqueous environment. Solubility and release profile of free base or free acid form of deterrent agent can be changed by adding pH modifying agent.

In preferred embodiment, dosage form may comprise at least one deterrent agent or combination of two or more deterrent agent of same category or of different category.

In some embodiment, antagonist agent can be used as deterrent agent to antagonize the effect of drug substance upon incorrect administration of dosage form. Selection of the antagonist agent is based on drug substance used in dosage form and it may vary according to therapeutic classes of drug substance. Antagonists useful in the present invention include, but not limited to, antagonists for opioids, non-opioid, central nervous system (CNS) depressants, stimulants, tranquilizers, barbiturates, hormones, cannabinoids, nicotine-like compounds, cold and cough drugs such as pseudoephedrine, glutamate antagonists, N-methyl-D-aspartate (NMDA) antagonists, etc. Suitable antagonist agents include, but are not limited to, naloxone, naltrexone, nalmefene, nalide, nalmexone, nalorphine, naluphine, haloperidol, promethazine, fluphenazine, perphenazine, levomepromazine, cyclazocine, thioridazine, perazine, chlorpromazine, chlorprothixine, zuclopentixol, flupentixol, prothipendyl, zotepine, benperidol, pipamperone, melperone, bromperidol and like.

In some embodiment, emetic agent can be used as deterrent agent. Emetics agent are agents which produce vomiting or emesis, which may be used in accordance with the invention as deterrent agent. Local or gastric emetics are the more rapid in their action, producing emesis in from two to five minutes. The systemic emetics must be absorbed and pass to the medulla before they produce vomiting, consequently requiring more time to exert their influence. Suitable emetic agents include, but not limited to, ipecacuanha, apomorphine, tartar emetic, zinc sulphate, copper sulphate, sodium chloride, mustard, ammonium carbonate and like.

In some embodiment, flushing agent can be used as deterrent agent in dosage form. For example, niacin, if taken in excessive dose, it produces warmth, flushing, and other uncomfortable symptoms.

In some embodiment, capsaicin can be used as irritating or burning agent in dosage form as deterrent agent. Capsaicin, if taken in excessive amount, it produces burning like feeling inside the stomach.

### pH Dependent Soluble Ingredient—

In some embodiment, an abuse deterrent pharmaceutical composition comprises pH dependent soluble ingredient. In particular, the pH-dependent soluble ingredient means ingredient whose characteristics, such as chemical and/or physical properties, vary according to the pH of the surrounding environment. The surrounding environment may comprise any type of liquid medium, such as gastrointestinal fluid, water, acid solution, alkaline solution, and like.

In some embodiments, a pH dependent soluble ingredient is included in a pharmaceutical composition in a form of coating on deterrent agent and additionally on pH modifying agent or a form of particle matrix with deterrent agent and additionally with pH modifying agent. The pH dependent soluble ingredient may be included in the dosage form in an amount sufficient to form coating or matrix to perform its intended function. The pH dependent soluble ingredient may impact release of deterrent agent depending on the pH of surrounding environment, which is raised or maintained by the pH modifying agent as a function of the amount of the pharmaceutical composition ingested: when the pharmaceutical composition is ingested in an appropriate dosage amount, the pH modifying agent is not present in an amount to alter sufficiently gastrointestinal pH to desire level to release deterrent agent but when the pharmaceutical component is ingested in an excess amount, the pH modifying agent is present in an amount to alter the gastrointestinal pH to desire level to release deterrent agent in effective amount to produce its intended effect.

In some embodiments, pH dependent soluble ingredients is included in the pharmaceutical composition in an amount of about 0.5 wt % to about 65 wt %; about 1 wt % to about 55 wt %; about 2 wt % to about 45 wt % or about 3 wt % to about 35 wt % of total weight of dosage form.

In preferred embodiment, pH dependent soluble ingredients include ingredients which are soluble at pH of stomach fluid and ingredients which are soluble at pH of Intestinal fluid.

In some embodiment, pH dependent soluble ingredient which are soluble at pH of stomach fluid include, but are not to be limited, calcium carbonate, chitin, chitosan, di and tribasic calcium phosphate, magnesium hydroxide, casein, polymethacrylate or methacrylic acid and its derivative such as dimethylaminoethyl methacrylate, butyl methacrylate, methyl methacrylate, copolymer of diethylaminoethyl methacrylate and methyl methacrylate, for example, Eudragit EPO, Eudragit E100, Eudragit 12.5, Kollicoat smartseal and like. Ingredient which is soluble at pH of stomach fluid means ingredient which is soluble only at pH of stomach fluid or ingredients which is easily soluble at pH of stomach fluid but slightly or slowly dissolve above pH of stomach fluid, ingredient which is soluble at pH of stomach fluid but may be permeable above pH of stomach fluid or ingredient which are degrade or digested only by stomach enzyme. According to literature, pH of stomach fluid is approximately within 1 to 5 in fed and fasting condition.

In some embodiment, pH dependent soluble ingredient which are soluble at pH of intestinal fluid include, but are not limited, polymethacrylate such as eudragit S 100, eudragit L 100, eudragit L 100-55 or mixture thereof, cellulose esters such as cellulose acetate phthalate, cellulose acetate trimellitate, hydroxypropylmethyl cellulose acetate succinate, HPMC phthalate, polyvinyl derivative such as polyvinyl acetate phthalate, shellac, zein, amylase starch and starch derivatives and like. Ingredient which is soluble at pH of intestinal fluid means ingredient which is soluble only at pH of intestinal fluid or ingredients which is easily soluble at pH of intestinal fluid but slightly or slowly dissolve below pH of intestinal fluid or ingredient which is soluble at pH of intestinal fluid but may be permeable below pH of intestinal fluid or ingredient which are degrade or digested only by intestinal enzyme. According to literature, pH of intestine fluid is approximately within 5 to 9.

### pH Modifying Agent—

In some embodiment, an abuse deterrent pharmaceutical composition comprises pH modifying agent which include pH modifying basic agent or pH modifying acidic agent or both. In some embodiment, antacid may be used as pH modifying basic agent.

In some embodiments, a pH modifying agent may be included in an amount such that gastrointestinal pH is not affected or mildly affected when the dosage form is taken in appropriate therapeutic amounts, but gastrointestinal pH may be increase or decrease or increase followed by decrease or decrease followed by increase at desire level when the pharmaceutical composition is ingested in excess amounts. In other word, amount of pH modifying agent in single dose unit is in not in sufficient amount to modify pH of gastrointestinal fluid at desire level but pH modifying agent of multiple units is in sufficient amount to modify pH of gastrointestinal pH at desire level.

In some embodiments, pH modifying agent is included in the pharmaceutical composition in an amount of about 10 wt % to about 90 wt %; about 25 wt % to about 85 wt %; about 40 wt % to about 80 wt % or about 40 wt % to about 75 wt % of total weight of dosage form.

pH modifying basic agent include, but are not to be limited, magnesium oxide, meglumine, sodium oxide, sodium hydroxide, sodium bicarbonate, sodium potassium tartrate, bismuth aluminate, bismuth carbonate, bismuth subcarbonate, bismuth subgallate, bismuth subnitrate, potassium citrate, sodium citrate, sodium carbonate, potassium bicarbonate, potassium carbonate, calcium carbonate, calcium phosphate, dibasic calcium phosphate, dihydroxyaluminum aminoacetate, dihydroxyaluminum sodium carbonate, glycine, magnesium glycinate, magnesium hydroxide, magnesium carbonate, sodium borate, aluminum oxide, aluminum hydroxide, ammonium Carbonate, monoethanolamine, diethanolamine, triethanolamine, Potassium Hydroxide, Sodium Phosphate Dibasic, Trolamine, sodium potassium tartrate, tribasic sodium phosphate, tricalcium phosphate and like. pH modifying basic agent also include agents that inhibit acid secretion in stomach such as, but not to be limited, histamines receptor antagonists and proton pump inhibitors.

pH modifying acidic agent include, but are not to be limited, ascorbic acid, benzoic acid, boric acid, citric acid, EDTA and derivative thereof like disodium edetate, trisodium edetate, tetrasodium edetate, disodium calcium edetate and like, edetic acid, erythrobic acid, fumaric acid, lactic acid, lauric acid, linoleic acid, malic acid, alginic acid, myristic acid, oleic acid, palmitic acid, sorbic acid, succinic acid, tartaric acid and like. pH modifying acidic agent also include agents that stimulate acid secretion in stomach such as, but not to be limited. histamines receptor agonists.

In some embodiments, one ingredient may also act as both pH dependent soluble ingredient and pH modifying agent. Examples of such suitable ingredients include calcium carbonate, di and tribasic calcium phosphate, and magnesium hydroxide.

### Other Pharmaceutical Additives—

The present invention can also optionally include other ingredients to enhance dosage form manufacture from a pharmaceutical composition of the present invention and/or alter the release profile of drug from pharmaceutical composition of the present invention.

Some embodiment of present invention comprises controlled release excipients include, but not limited to, natural, synthetic or semi-synthetic polymer, cellulose derivatives, polymethacrylate or methacrylic acid derivatives, vinyl pyrrolidone and copolymers of vinyl pyrrolidone, polyalkylene oxide and copolymer thereof, polyacrylic acid, gums of plant, animal, mineral or synthetic origin, carageenan, polylactic acid or polyglycolic acid and copolymers thereof, polyvinyl alcohol, polyvinyl acetate and its copolymer, fatty acids, wax, fatty alcohol, lipid, steryl alcohol, oil, natural and synthetic glycerides, and like.

Some embodiment of present invention comprises one or more fillers or diluents such as cellulose derivatives, mono, di or tri basic calcium phosphate, sugar, starch derivatives, acid or base, and like.

Some embodiment of present invention comprises one or more disintegrant may be highly/rapidly swellable, moderately swellable or slowly swellable such as vinylpyrrolidone polymers, cellulose and cellulose derivatives, sodium starch glycolate, starch and starch derivatives, resins, and like.

Some embodiment of present invention may also comprise binder, glidant, lubricant, plasticizers, anti-adhesives, surfactants, colorants, flavoring agents, wiking agent or anti oxidant. Examples of such kind of excipients are described in Handbook of Pharmaceutical excipients, 7^{th} edition.

However, choice of other additives typically depends on the chemical and physical properties of the drug, desire drug release profile and design of dosage form.

As used herein, the term “about” is understood to mean ±5% of the value referenced. For example, “about 45%” is understood to literally mean 40% to 50%.

The dosage form can be in an immediate release form or a controlled release form or a combination of a controlled release form and an immediate release form.

According to the invention, dosage form can be a matricial type form or non matricial form. “Matricial type form” is intended to denote, in the present disclosure, a form in which the active substance and/or deterrent agent is dispersed in a solid continuous phase. Said matricial or non matricial type form e.g. consists of pharmaceutically acceptable additives known by the man skilled in the art. The matricial form element includes, for instance, a plurality of matricial granules, pellets, minitablets, spheres or beads of the active substance, deterrent agent or pH modifying agent while non matricial form element include, for instance, pellets, minitablets, spheres, spheroids or particles of the active substance, deterrent agent or pH modifying agent; wherein active substance and pH modifying agent are non-coated and/or coated by one or more film (for instance, controlled release film and/or pH dependent soluble film) while deterrent agent is coated by one or more film (controlled release film and/or pH dependent soluble film). The matricial type or non matricial type dosage form can be a tablet(s) or capsule.

According to the invention, dosage form can be a reservoir type form. “Reservoir type form”, is intended to denote, in the present disclosure, a form in which the volume of material containing the active substance and/or deterrent agent is entirely coated by one or more film (controlled release film and/or pH dependent soluble film) which controls the diffusion release rate of the active substance and/or deterrent agent through the continuous film. This release occurs as a result of the contact of the system with the liquid of the gastro intestinal tract. The material containing active substance or deterrent agent is, for example, the active substance or deterrent agent as such, a mixture of pharmaceutical additives with the active substance or deterrent agent, or active substance or deterrent agent coated on pharmaceutical additive such as spheres, pellets, beads or mini or micro tablet or capsule. Controlled release film comprises hydrophobic material, hydrophilic material, pH dependent soluble material or any combination thereof. The reservoir form also comprises pH modifying agent with or without coating of pH dependent soluble ingredient. The reservoir form can be an individually coated tablet, capsule or multiparticulates form such as microtablets or minitablets, micropellets, granules, spheroids, beads, pellets or particles. Multiparticulates can be packed in capsule or compressed in tablet form using other suitable pharmaceutical additives.

According to present invention, an abuse deterrent pharmaceutical composition can be formulated in form of, but not limited to, tablet, microtablets or minitablets, micropellets, granules, spheroids, beads or pellets, capsule, powders, caplets, pills, suppositories, gels, soft gelatin capsules, capsules and compressed tablets manufactured from a pharmaceutical composition of the present invention.

According to present invention, an abuse deterrent pharmaceutical composition can be formulated in form of liquid or semisolid using suitable pharmaceutically additives.

### EXAMPLES

Certain aspects of the present invention may be better understood as illustrated by the following examples, which are meant by way of illustration and not limitation.

### Example 1

According to molecular mass, 1000 mg of potassium bicarbonate is sufficient to increase pH of 1000 ml 0.01N HCl above 6. If single dosage unit comprise 350 mg of potassium bicarbonate, then three dosage units are require to increase pH of 1000 ml of 0.01N HCl above 6 because three dosage units comprises total 1050 mg of potassium bicarbonate. pH of 0.01N HCl is around 2 which is similar to pH of stomach fluid.

In two beakers, 900 ml of 0.01N HCl was taken and pH was measure. pH of 0.01N HCl in both beaker was 1.90. According to literature, hypromellose phthalate 55 is dissolved at pH 5.5 or it above. 500 mg of hypromellose phthalate 55 was added in both beaker and stirred for 20 minutes. However, after 20 minutes, hypromellose phthalate 55 was appeared as undissolved in both beakers. 350 mg of potassium bicarbonate equivalent to single dosage unit was added in beaker 1 while of potassium bicarbonate equivalent to three dosage units were added in beaker 2. After 20 minutes continues stirring, hypromellose phthalate 55 was appeared as undissolved in beaker 1 while it was dissolved in beaker 2 because elevation of pH of media of beaker 2. After addition of potassium bicarbonate, pH of media of beaker 1 was 2.30 which is similar to pH of stomach fluid while pH of media of beaker 2 was 6.15 which is similar to pH of intestinal fluid.

### Example 2

Similar to above example, in two beaker, 500 ml of 0.01N HCl was taken. In beaker 1, one enteric coated tablet of Rabeprazole sodium was added and in beaker 2, three enteric coated tablets of Rabeprazole sodium were added and stirred for 20 minutes. However, in both beakers, tablets were remained intact after 20 minutes continued stirring. 70 mg of magnesium oxide and 55 mg of sodium carbonate equivalent to single tablets was added in beaker 1 while 210 mg of magnesium oxide and 165 mg of sodium carbonate equivalent to three tablets were added in beaker 2. After 20 minutes continued stirring, tablet in beaker 1 was remained intact while tablets in beaker 2 were started to disintegrate, which indicate that enteric coating surrounding tablets was dissolved because elevation of pH of media of beaker 2.

1. (canceled)

2. (canceled)

3. (canceled)

4. (canceled)

5. (canceled)

6. (canceled)

7. (canceled)

8. (canceled)

9. (canceled)

10. (canceled)

11. An abuse deterrent pharmaceutical composition comprising:
a. active ingredient;
b. deterrent agent;
c. pH dependent soluble ingredient; and
d. pH modifying agent;
wherein the composition have the following features:
I. alter the solubility of the pH dependent soluble ingredient when the composition ingested in excess of an intended dosage; and
II. release the deterrent agent in effective amount to produce its intended aversive effect when the composition administered after tampering.

12. An abuse deterrent pharmaceutical composition comprising:
a. active ingredient;
b. deterrent agent;
c. pH dependent soluble ingredient;
d. pH modifying agent; and
e. controlled release excipient;
wherein the pH modifying agent alter the solubility of the pH dependent soluble ingredient when the composition ingested in excess of an intended dosage;
wherein the deterrent agent release in effective amount to produce its intended aversive effect when the composition administered incorrectly.

13. An abuse deterrent pharmaceutical composition in reservoir form comprising active substance particulates, deterrent agent particulates and pH modifying agent;
wherein the active substance particulate comprising
i. active ingredient with or without pharmaceutical additive, and
ii. at least one of controlled release film and pH dependent soluble film surrounding the step (i) active ingredient;
wherein the deterrent agent particulate comprising
i. deterrent agent with or without pharmaceutical additive, and
ii. at least one of controlled release film and pH dependent soluble film surrounding the step (i) deterrent agent;
wherein the pH modifying agent alter the solubility of the pH dependent soluble ingredient when the composition ingested in excess of an intended dosage.

14. An abuse deterrent pharmaceutical composition of any of claim 11, claim 12 and claim 13 is in unit dosage form, wherein the unit dosage form include tablet, capsule, liquid, semisolid or multiparticulates packed in unit dose container or compressed to form tablet, wherein multiparticulates include powder, granules, particles, pellets, beads, spheres, spheroid, particulates, minitablets and like.

15. An abuse deterrent pharmaceutical composition of claim 11 or claim 12, wherein the active ingredient is
coated by at least one of pH dependent soluble ingredient and controlled release film or
contained within matrix of at least one of pH dependent soluble ingredient and controlled release excipients.

16. An abuse deterrent pharmaceutical composition of claim 11 or claim 12, wherein the deterrent agent is
coated by at least one of pH dependent soluble ingredient and controlled release excipients or
contained within matrix of at least one of pH dependent soluble ingredient and controlled release excipients.

17. An abuse deterrent pharmaceutical composition of any of claim 11, claim 12 and claim 13, wherein the pH modifying agent comprises acidic agent, or basic agent, or combination thereof

18. An abuse deterrent pharmaceutical composition of any of claim 11, claim 12 and claim 13, wherein pH modifying agent is present in amount of about 10% w/w to about 90% w/w of the composition.

19. An abuse deterrent pharmaceutical composition of any of claim 11, claim 12 and claim 13, wherein pH modifying acidic agent coated by pH dependent soluble ingredient or contained within matrix of pH dependent soluble ingredient.

20. An abuse deterrent pharmaceutical composition of any of claim 11, claim 12 and claim 13, wherein the amount of the pH modifying agent not alter the solubility of the pH dependent soluble ingredient when the composition ingested in an intended dosage but the amount of the pH modifying agent alter the solubility of the pH dependent soluble ingredient when the composition ingested in excess of an intended dosage within time period of less than one hour.

21. An abuse deterrent pharmaceutical composition of any of claim 11, claim 12 and claim 13, wherein the pH modifying agent alter the solubility of the pH dependent soluble ingredient when greater than two unit doses ingested within time period of less than one hour.

22. An abuse deterrent pharmaceutical composition of any of claim 11, claim 12 and claim 13, wherein pH dependent soluble ingredient is present in an amount of about 0.5% w/w to about 65% w/w of the composition.

23. An abuse deterrent pharmaceutical composition of any of claim 11, claim 12 and claim 13, wherein the pH dependent soluble ingredient has any of following characteristics:
a. soluble at a pH of stomach fluid but substantially insoluble at a pH of intestinal fluid or
soluble at pH value of less than about 5 but substantially insoluble at pH value of greater than about 5 or
degradable by enzyme of stomach fluid but substantially not degradable by enzyme of intestinal fluid;
b. soluble at a pH of intestinal fluid but substantially insoluble at a pH of stomach fluid or
soluble at pH value of greater than about 5 but substantially insoluble at pH value of less than about 5 or
degradable by enzyme of intestinal fluid but substantially not degradable by enzyme of stomach fluid.

24. An abuse deterrent pharmaceutical composition of any of claim 11, claim 12 and claim 13, wherein the controlled release excipients comprises one or more of natural or synthetic polymer; cellulose derivatives; polymethacrylate or methacrylic acid derivatives; vinyl pyrrolidone and copolymers of vinyl pyrrolidone; polyalkylene oxide and copolymer thereof; polyacrylic acid; gums of plant, animal, mineral or synthetic origin; carageenan; polylactic acid or polyglycolic acid and copolymers thereof; polyvinyl alcohol; polyvinyl acetate and its copolymer; fatty acids; wax; fatty alcohol; lipid; steryl alcohol; oil; natural and synthetic glycerides; or combinations thereof

25. An abuse deterrent pharmaceutical composition of any of claim 11, claim 12 and claim 13, wherein the composition have following features:
a. the pH modifying agent alter the solubility of the pH dependent soluble ingredient when greater than two unit doses ingested within time period of less
